(19) United States
(12) Patent Application Publication
(10) Pub. No.: US 2013/0110056 A1
(43) Pub. Date: May 2, 2013

(54) Neurosurgical Sponge Apparatus with Dissolvable Layer

(71) Applicant: American Surgical Sponges, LLC, Lynn, MA (US)

(72) Inventor: Matthew Gombrich, Underhill, VT (US)

(73) Assignee: American Surgical Sponges, LLC, Lynn, MA (US)

(21) Appl. No.: 13/661,586

(22) Filed: Oct. 26, 2012

Related U.S. Application Data
(60) Provisional application No. 61/552,803, filed on Oct. 28, 2011.

(51) Int. Cl. A61F 13/40

(52) U.S. Cl. 604/290; 604/304

(57) ABSTRACT

A neurosurgical sponge apparatus and related methods are disclosed. The neurosurgical sponge apparatus has a first layer formed from a fluid absorbent material. A second layer is affixed to the first layer, wherein the second layer is formed from a water-soluble material.

### CROSS REFERENCE TO RELATED APPLICATION

This application claims benefit of U.S. Provisional Application Ser. No. 61/552,803 entitled, “Neurosurgical Sponge Apparatus with Dissolvable Layer” filed Oct. 28, 2011, the entire disclosure of which is incorporated herein by reference.

### FIELD OF THE DISCLOSURE

The present disclosure is generally related to medical sponges and more particularly is related to a neurosurgical sponge apparatus with dissolvable layer.

### BACKGROUND OF THE DISCLOSURE

Many surgeries require the use of sponges placed over surgical areas for various purposes. For example, in some surgeries, such as those around the brain or other critical structures, sponges are used to retain moisture within the surgical area and on the tissue. This especially is common for cranial surgeries where portions of a patient's brain may be exposed. A sponge may be draped over the exposed portions of the brain to prevent excessive drying or exposure to the air. The need for a sponge to prevent drying of the tissue may be heightened with lengthy surgeries or surgeries that require repeated exposure of the tissue from a sponge. With surgeries lasting many hours, it may be necessary for many sponges to be used in a successive manner, where a used sponge is replaced with a new sponge after the used sponge has provided the extent of its benefits.

Although the sponge may be successful in retaining moisture within the surgical area, use of the sponge may present complications with the surgery. For example, when the sponge is to be removed, it may have adhered to the tissue within the surgical area that it has been in contact with. When the sponge is pulled away from the tissue, portions of the neuronal tissue from the brain or other surgical site may be removed with the sponge, since they are apt to become adhered to the porous or fibrous materials of the sponge. Removing even a small quantity of tissue from a critical surgical site may have detrimental consequences to the patient, since the disrupted tissue can cause medical damage to the patient or necessitate additional medical procedures.

Thus, a heretofore unaddressed need exists in the industry to address the aforementioned deficiencies and inadequacies.

### SUMMARY OF THE DISCLOSURE

Embodiments of the present disclosure provide a system and method for a neurosurgical sponge apparatus. Briefly described, in architecture, one embodiment of the system, among others, can be implemented as follows. The neurosurgical sponge apparatus comprises a first layer formed from a fluid absorbent material. A second layer is affixed to the first layer, wherein the second layer is formed from a water-soluble material.

The present disclosure can also be viewed as providing methods of using a medical sponge. In this regard, one embodiment of such a method, among others, can be broadly summarized by the following steps: placing a neurosurgical sponge apparatus on a quantity of neuronal tissue, wherein the neurosurgical sponge apparatus has a first layer formed from a fluid absorbent material and a second layer affixed to the first layer, wherein the second layer is formed from a water-soluble material; dissolving the second layer on the quantity of neuronal tissue; and removing the neurosurgical sponge apparatus with substantially no damage to the quantity of neuronal tissue.

The present disclosure can also be viewed as providing a neurosurgical sponge apparatus. Briefly described, in architecture, one embodiment of the apparatus, among others, can be implemented as follows. A moistened first layer is formed from a fluid-retaining fabric. A second layer is affixed abutting to a first side of the first layer, wherein the second layer is formed from at least one water-soluble material. A low-adhesion barrier is formed by the second layer when the second layer is contacted by a quantity of neuronal tissue after a predetermined length of time.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a cross-sectional illustration of a neurosurgical sponge apparatus, in accordance with a first exemplary embodiment of the present disclosure.

FIG. 2 is a cross-sectional illustration of a neurosurgical sponge apparatus, in accordance with a second exemplary embodiment of the present disclosure.

FIG. 3 is a plan view illustration of a neurosurgical sponge apparatus, in accordance with a third exemplary embodiment of the present disclosure.

FIG. 4 is a cross-sectional side view of a neurosurgical sponge apparatus, in accordance with a fourth exemplary embodiment of the present disclosure.

FIG. 5 is a flowchart illustrating a method of using a medical sponge apparatus in accordance with the first exemplary embodiment of the disclosure.

### DETAILED DESCRIPTION

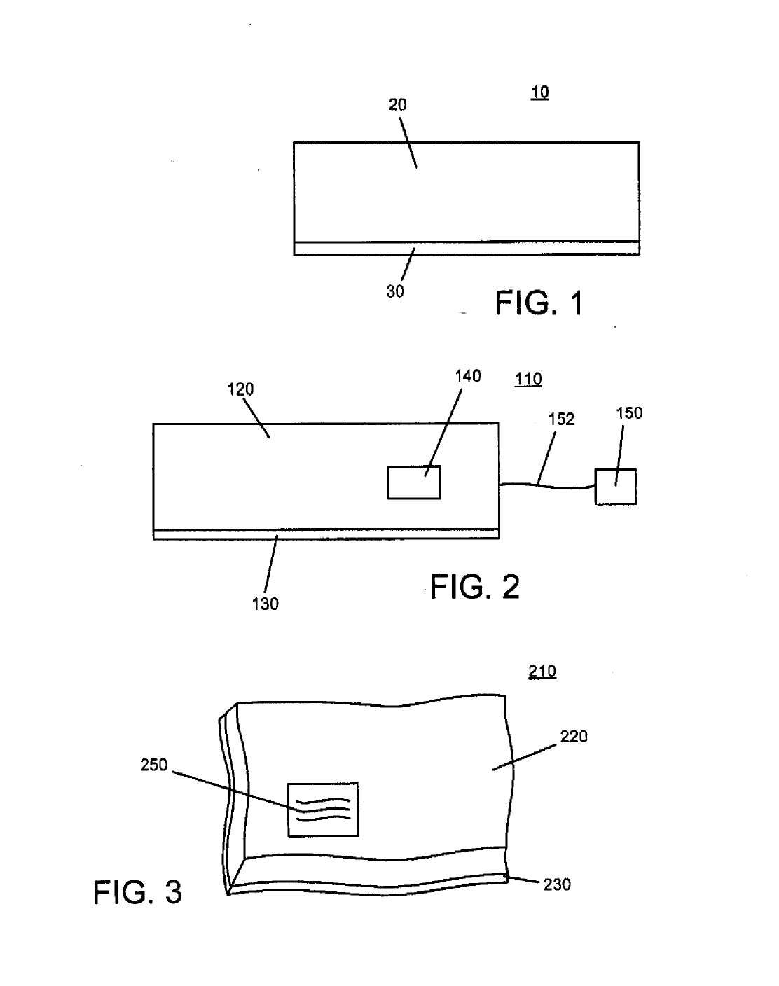

FIG. 1 is a cross-sectional illustration of a neurosurgical sponge apparatus 10, in accordance with a first exemplary embodiment of the present disclosure. The neurosurgical sponge apparatus 10, which may be referred to herein simply as ‘apparatus 10’ includes a first layer 20 formed from a fluid absorbent material. A second layer 30 is affixed to the first layer 20, wherein the second layer 30 is formed from a water-soluble material.

The neurosurgical sponge apparatus 10 may be used in any medical procedure in any medical field or setting, including surgeries, examinations, and operations on any human being or other living being. Commonly, the apparatus 10 may be used in surgeries or operations that are prone to neuro-rupture such as critical areas having a significant quantity of neuronal tissue, like a brain. However, the apparatus 10 may also be used proximate to other types of tissue, or other parts of a patient's body, such as proximate to blood vessels or other organs, such as the eyes, ears, or mouth. The neurosurgical sponge apparatus 10 may have a particularly applicable use in cranial surgeries where areas of a patient's brain regularly are covered with a medical sponge.

The apparatus 10 may provide a variety of benefits when used with a surgical procedure or other medical operation. In particular, the apparatus 10 may be used to assist with keeping exposed neuronal tissue moist before, after, and during a surgical operation. During extended surgical procedures, exposed neuronal tissue located near a surgical site may experience excessive drying which may cause complications with the surgical procedure, unwanted medical harm to the patient, and even the risk of fatalities. To prevent excessive drying of the tissue, the apparatus 10 may be moistened and applied to the tissue during the course of the surgical procedure. The apparatus 10 may also offer other benefits within the medical field, including preventing contact of the tissue or critical areas, such as blood vessels and organs, from a surgical tool, or by absorbing fluid from the tissue, the critical area, or a proximate surgical area. For any type of use, the apparatus 10 may provide significant benefits when it comes to removing a used apparatus 10 from the neuronal tissue, the critical area, or any other surgical site. As discussed herein, the apparatus 10 may have low adhesion properties between itself and the tissue, critical area, or surgical site that it was contacting, thus allowing the apparatus 10 to be removed with substantially no damage or disruption to the tissue, critical area, or surgical site.

The first layer 20 of the neurosurgical sponge apparatus 10 may include any type of fluid-retaining or fluid-absorbent, biocompatible material, which may include any material that is conventionally used in medical sponges, such as absorbent or hydrating fibrous materials. For example, the first layer 20 may include a pliable, solid, fluid-absorbing scaffold or patty that is constructed from cotton, rayon, polyester, or any mixture of absorbent materials. The first layer 20 is biocompatible, so it can be used on a patient with an opening in their body without causing harm to the patient, or creating a negative reaction within the patient. For example, the absorbent, biocompatible material may include a cotton woven or non-woven material, with any number of plys and fabrics. Other materials may include any combination of polyester, cellulose, foam, or any other absorbent material, all of which are considered within the scope of the present disclosure.

The second layer 30 is affixed directly on, abutting, or proximate to at least one side of the first layer 20, such that the first and second layers 20, 30 form a substantially unitary structure. The second layer 30 may be formed from a water-soluble material that is dissolvable when contacted to the tissue, critical area, or surgical site. For example, the second layer 30 may be a biologically inert dissolvable neural surface polymer matrix that can be placed in direct contact with the open tissue of the surgical area. Thus, the second layer 30 may dissolve into a viscous fluid that at least partially contacts and is integrated into the tissue of the surgical area. Therefore, the second layer 30 formed with the water-soluble material must be biocompatible. The dissolution of the second layer 30 may be initiated by contact with moist tissue of the patient or other fluid of the patient, such as blood. When the second layer 30 dissolves, it may form a low adhesion barrier layer between the first layer 20 of the apparatus 10 and the tissue that the apparatus 10 contacts. This low adhesion barrier layer may have a range of viscosities and may be viscous enough to retain the apparatus 10 on the tissue, yet still allow the first layer 20 to be removed from the tissue without adhering to the tissue to the point where the tissue material is subject to damage or degradation.

The second layer 30 may be constructed from a number of materials, some of which may be organic materials and some of which may be inorganic materials. For example, the second layer 30 may be constructed from gelatin, collagen, cellulose, pullulan, hydroxypropylmethyl cellulose, hydroxyethyl cellulose, chitin, chitosan, amylose, dextran, polyethylene glycol, phospholipid phospholipid particles or entities, polyethylene oxide, polyacrylic acid, pectin, or any combination thereof. In some uses of the apparatus 10, the second layer 30 may be formed from two or more water-soluble materials. The type of water-soluble material(s) that is used within the apparatus 10 may be selected based on a desired characteristic of the apparatus 10, such as a solubility factor of the second layer 30 or a dissolution time of the second layer 30. Similarly, the second layer 30 may dissolve at specific, predetermined temperatures. For example, it may be preferable for the second layer 30 to dissolve at temperatures between 35° C. to 40° C. However, the second layer 30 may be designed to dissolve at any other temperature, as may vary based on design of the apparatus 10 and/or the intended use of the apparatus 10.

The first and second layers 20, 30 of the apparatus 10 may be selected or manufactured based on certain desired characteristics of the apparatus 10. For instance, the first layer 20 may have a predetermined fluid-retention factor which may be characterized as a measurement or designation of the ability of the first layer 20 to retain fluid, which may be achieved through the material(s) forming the first layer 20, a thickness of the first layer 20, or another characteristic of the first layer 20. The second layer 30 may have a predetermined solubility factor which may be characterized as a measurement or designation of the ability of the second layer 30 to dissolve, which may be achieved through water-soluble material selection, size, application, or another characteristic of the second layer 30. The predetermined fluid-retention factor of the first layer 20 may be matched or selected to correspond, exactly or substantially, with the predetermined solubility factor of the second layer 30.

For example, the first layer 20 may have a predetermined fluid-retention factor that allows the first layer 20 to properly retain fluid on neuronal tissue for 60 minutes. After 60 minutes, the apparatus 10 must be replaced to provide the proper level of hydration on the neuronal tissue. Accordingly, the second layer 30 may be selected to have a predetermined solubility factor that initiates dissolution of the second layer 30 after 45 minutes of contact with the neuronal tissue, such that at the 60 minute point, the second layer 30 has sufficiently dissolved into the low adhesion barrier between the first layer 20 and the neuronal tissue and the apparatus 10 may be removed without damage to the neuronal tissue. Although the above example is non-limiting to the scope of the present disclosure, it can be seen that the characteristics of the first and second layers 20, 30 may be matched to allow for predictable and efficient use of the apparatus 10.

The predetermined solubility factor of the second layer 30 may be achieved, in one example, providing the second layer 30 with a predetermined thickness, i.e., where the water-soluble material forming the second layer 30 is constructed as a polymer film with a predetermined thickness. The thickness of the polymer film may correspond to the predetermined fluid-retention factor of the first layer 20, such as a fluid-retention time or an absorption ability of the first layer 20. The various characteristics of the first and second layers 20, 30 may vary widely. For example, the first layer 20 may have the ability to retain fluid for any length of time, including 30 minutes or less, 60 minutes, 120 minutes or longer times, or any other period of time. Similarly, the second layer 30 may have the ability to dissolve at any point in time, such as 30 minutes or less, 60 minutes, 120 minutes or longer times, or any other period of time after contact with the tissue. This ability of the first and second layers 20, 30 to be used with specific, known lengths of time may allow the apparatus 10 to be used properly for surgical procedures with different lengths with substantially corresponding degradation times. Similar to the ability of the apparatus 10 to be used for different periods of time, the apparatus 10 may also be keyed or designed for use with specific materials, in certain operating conditions, for particular types of tissue, and/or for use with other surgical items, such as rotary drills or sharp objects.

The second layer 30 may have a number of variations and designs. For example, the second layer 30 may be impregnated with a biologically active compound. This may include impregnation of the second layer 30 with epinepherine, naropine, bupivacaine, mepivacaine, and/or pro-coagulants, such as, thrombin. Any other type of biologically active compound may also be used. The total mass of the neurosurgical sponge apparatus 10 may include any amount of either of the first or second layers 20, 30. For example, the neurosurgical sponge apparatus 10 may include approximately 1-5% of the second layer 30 to 95-99% of the first layer 20. In other examples, the neurosurgical sponge apparatus 10 may include a second layer 30 having 5-10%, 10-15%, 15-20%, or any other percentage of the total neurosurgical sponge apparatus 10 composition or mass.

The combination of the first and second layers 20, 30 forming the apparatus 10 may result in a structure that promotes both fluid-retention and/or absorbance of fluid and ultra-low adhesion properties with neuronal tissue. Accordingly, the apparatus 10 may be used in surgical procedures to effectively moisten the neuronal tissue by retaining fluid in contact with the tissue during the surgical operation, as well as be easily removed without damaging neuronal tissue to which the apparatus 10 has been placed in contact with. When the apparatus 10 is removed from the tissue, the apparatus 10 via will have very little or no negative effect on the neuronal tissue it was contacting since the second layer 30 will dissolve during the operation to create an ultra-low adhesion barrier to the neuronal tissue. The apparatus 10 may be used in any type of surgery or medical procedure, but it may be particularly applicable for neurosurgical procedures where neuronal tissue is exposed to a working environment.

FIG. 2 is a cross-sectional illustration of the neurosurgical sponge apparatus 110, in accordance with a second exemplary embodiment of the present disclosure. The neurosurgical sponge apparatus 110 of FIG. 2, which is referred to herein simply as ‘apparatus 110’ may include any of the features discussed with respect to any of the embodiments of this disclosure. The apparatus 110 includes a radiopaque marker 140 which may be embedded or otherwise affixed to the apparatus 110, such as by being embedded within or affixed to the first layer 120. The radiopaque marker 140 may include any material that substantially prevents radiation penetration, such as radiation from X-rays. As one having skill in the art would recognize, this allows the surgeon to account for all apparatus 110 used within a particular medical operation, thereby preventing any accidental situations where the apparatus 110 is left within a patient's body after the surgical procedure.

Also shown in FIG. 2 is a positioning element 152 attached to the apparatus 110. The positioning element 152 may include any type of positioning structure, such as flexible string or a substantially rigid tab (not shown) constructed from a medically safe material. The positioning element 152 may allow the surgeon or medical assistant to position, reposition, or locate the neurosurgical sponge apparatus 110 as needed. It may also allow for removal of the neurosurgical sponge apparatus 110. The positioning element 152 may include any design, may be any size, and may be affixed to the neurosurgical sponge apparatus 110 in any configuration. For example, the positioning element 152 may be affixed to an interior portion of the neurosurgical sponge apparatus 110, such as between the first layer 120 and the second layer 130, as is shown in FIG. 2, or it may be affixed to the exterior of the apparatus 110.

An identification element 150 may also be included with the apparatus 110 to allow for identification of a characteristic or property of the sponge. The identification element 150 may be affixed to the positioning element 152, as is shown in FIG. 2, or it may be affixed elsewhere on the apparatus 110, as is discussed with respect to FIG. 3. The identification element 150 may include any serial number or character(s), any color, any design, or other identifying mark. For example, the identification element 150 may be a brightly colored marker that easily shows the position of the apparatus 110. The identification element 150 may also indicate the number of apparatus 110 used in a procedure, i.e., by indicating that the apparatus 110 is ‘sponge 7 of 10,’ or the type of neurosurgical sponge apparatus 110, such as the fluid retention, absorption, or dissolution properties of the apparatus 110. Of course, other features and components that are commonly found in use with conventional sponges may also be used with the apparatus 110, all of which are considered within the present disclosure.

FIG. 3 is a plan view illustration of a neurosurgical sponge apparatus 210, in accordance with a third exemplary embodiment of the present disclosure. The neurosurgical sponge apparatus 210 of FIG. 3, which may be referred to herein simply as ‘apparatus 210’ may include any of the features discussed with respect any of the embodiments of this disclosure. The apparatus 210 is shown with the identification element 250 positioned on the exterior of the apparatus 210. Accordingly, the identification element 250 may be positioned on any surface of the apparatus 210, generally on the surface of the first layer 220 since the second layer 230 will dissolve during the surgical procedure.

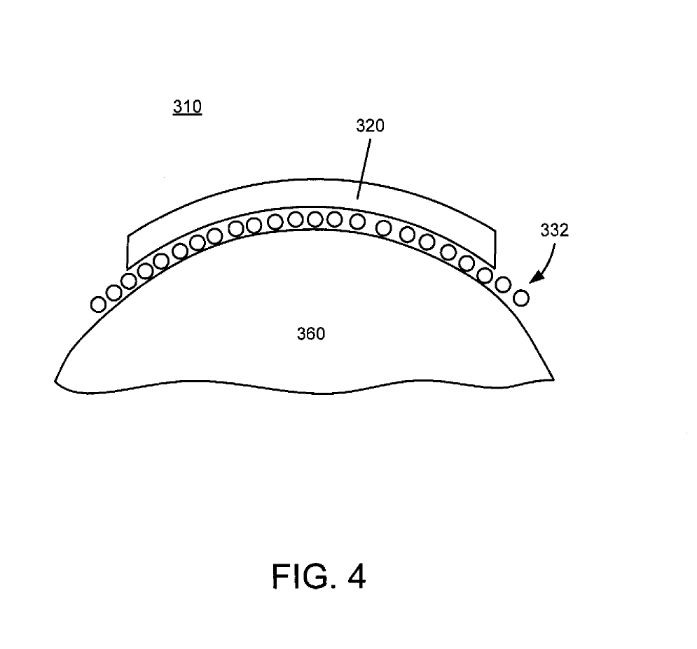

FIG. 4 is a cross-sectional side view of a neurosurgical sponge apparatus 310, in accordance with a fourth exemplary embodiment of the present disclosure. The neurosurgical sponge apparatus 310 of FIG. 4, which may be referred to herein simply as ‘apparatus 310’ may include any of the features discussed with respect any of the embodiments of this disclosure. In particular, FIG. 4 depicts the apparatus 310 in use with a quantity a neuronal tissue 360 after the apparatus 310 has been positioned in contact with the neuronal tissue 360 for a period of time. As can be seen, the second layer (not shown) may be dissolved into the low adhesion barrier 332 which is positioned between the first layer 320 and the neuronal tissue 360. The low adhesion barrier 332 may retain the first layer 320 in place on the neuronal tissue 360 yet still allow for the first layer 320 to be removed by the surgeon or medical technician. Accordingly, when the first layer 320 is peeled away from the neuronal tissue 360, the low adhesion barrier 332 may allow separation of the first layer 320 from the neuronal tissue 360 through the low adhesion barrier 332. After removal of the apparatus 310, small quantities of remaining fluid-soluble material that formed the low adhesion barrier 332 may remain on both the first layer 320 and the neuronal tissue 360.

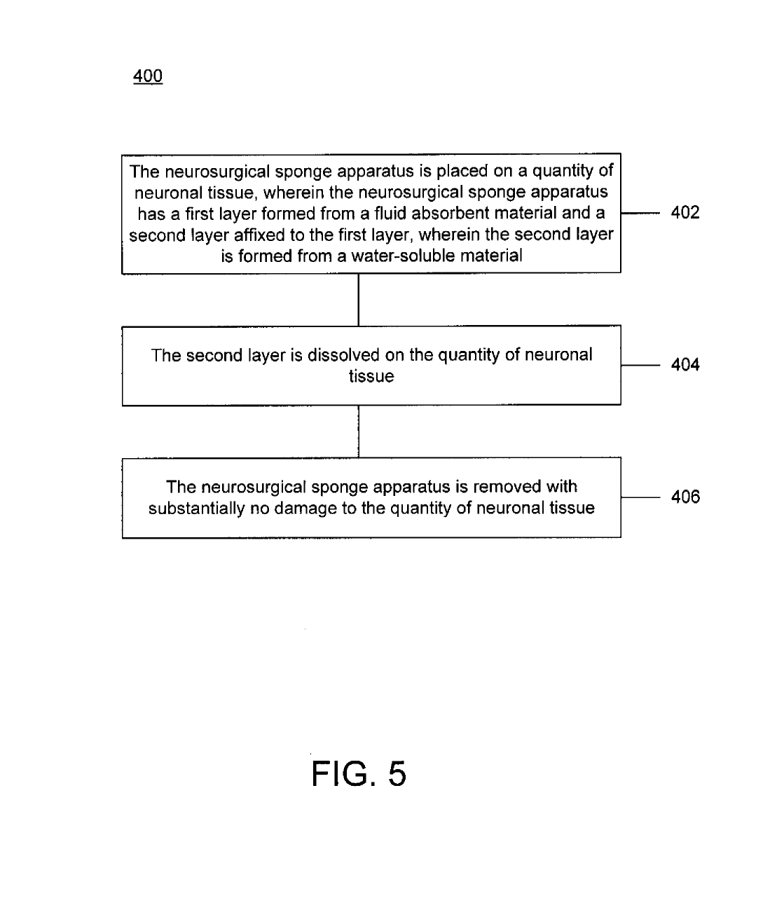

FIG. 5 is a flowchart 400 illustrating a method of using a medical sponge apparatus in accordance with the first exemplary embodiment of the disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 402, a neurosurgical sponge apparatus is placed on a quantity of neuronal tissue, wherein the neurosurgical sponge apparatus has a first layer formed from a fluid absorbent material and a second layer affixed to the first layer, wherein the second layer is formed from a water-soluble material. The second layer is dissolved on the quantity of neuronal tissue (block 404). The neurosurgical sponge apparatus is removed with substantially no damage to the quantity of neuronal tissue (block 406).

The method may include any additional number of steps, processes, functions, or variations thereof, including any of the functions described relative to FIGS. 1-4. For example, the second layer may be dissolved at a predetermined temperature, such as a temperature of between 35° C. to 40° C. When the second layer dissolves, a low adhesion barrier layer may be formed between the first layer and the quantity of neuronal tissue. A dissolution time of the second layer may be substantially matched with a predetermined fluid-retention factor of the first layer. When the neurosurgical sponge apparatus is used to retain moisture or fluid proximate to the neuronal tissue, the first layer may be moistened prior to or after placing the neurosurgical sponge apparatus on the quantity of neuronal tissue.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and the present disclosure and protected by the following claims.

What is claimed is:

1. A neurosurgical sponge apparatus comprising:
a first layer formed from a fluid absorbent material; and
a second layer affixed to the first layer, wherein the second layer is formed from a water-soluble material.

2. The neurosurgical sponge apparatus of claim 1, further comprising at least one radiopaque marker affixed to the first layer.

3. The neurosurgical sponge apparatus of claim 1, further comprising at least one identification element affixed to the first layer.

4. The neurosurgical sponge apparatus of claim 3, wherein the at least one identification element is affixed to the first layer with at least one positioning element.

5. The neurosurgical sponge apparatus of claim 1, wherein the water-soluble material of the second layer further comprises at least one of: gelatin, collagen, cellulose, pullulan, hydroxypropylmethyl cellulose, hydroxyethyl cellulose, chitin, chitosan, amylose, dextran, polyethylene glycol, phospholipid particles, polyethylene oxide, polyacrylic acid, and pectin.

6. The neurosurgical sponge apparatus of claim 1, wherein the water-soluble material of the second layer further comprises a biologically inert dissolvable neural surface polymer matrix.

7. The neurosurgical sponge apparatus of claim 1, wherein the second layer is formed from at least two water-soluble materials.

8. The neurosurgical sponge apparatus of claim 1, wherein the second layer substantially dissolves when contacted by a quantity of neuronal tissue.

9. The neurosurgical sponge apparatus of claim 8, wherein upon contact by the quantity of neuronal tissue, the second layer forms a low adhesion barrier layer between the first layer and the quantity of neuronal tissue.

10. The neurosurgical sponge apparatus of claim 8, wherein the second layer substantially dissolves between 35° C. to 40° C.

11. The neurosurgical sponge apparatus of claim 1, wherein the first layer has a predetermined fluid-retention factor and the second layer has a predetermined solubility factor, wherein the predetermined solubility factor of the second layer is selected to substantially correspond to the predetermined fluid-retention factor of the first layer.

12. The neurosurgical sponge apparatus of claim 11, wherein the predetermined fluid-retention factor of the first layer is determined by a thickness dimension of the first layer and the predetermined solubility factor of the second layer is a length of dissolution time of the second layer.

13. The neurosurgical sponge apparatus of claim 11, wherein the predetermined fluid-retention factor of the first layer is determined by a moistening ability of the first layer and the predetermined solubility factor of the second layer is a length of dissolution time of the second layer.

14. A method of using a medical sponge comprising:
placing a neurosurgical sponge apparatus on a quantity of neuronal tissue, wherein the neurosurgical sponge apparatus has a first layer formed from a fluid absorbent material and a second layer affixed to the first layer, wherein the second layer is formed from a water-soluble material;
dissolving the second layer on the quantity of neuronal tissue; and
removing the neurosurgical sponge apparatus with substantially no damage to the quantity of neuronal tissue.

15. The method of claim 14, wherein the step of dissolving the second layer on the quantity of neuronal tissue further comprises dissolving the second layer at a temperature of between 35° C. to 40° C.

16. The method of claim 14, wherein the step of dissolving the second layer on the quantity of neuronal tissue further comprising forming a low adhesion barrier layer between the first layer and the quantity of neuronal tissue.

17. The method of claim 14, further comprising the step of forming the water-soluble material of the second layer with at least one of: gelatin, collagen, cellulose, pullulan, hydroxypropylmethyl cellulose, hydroxyethyl cellulose, chitin, chitosan, amylose, dextran, polyethylene glycol, phospholipid particles, polyethylene oxide, polyacrylic acid, and pectin

18. The method of claim 14, wherein the step of dissolving the second layer on the quantity of neuronal tissue further comprises substantially matching a dissolution time of the second layer with a predetermined fluid-retention factor of the first layer.

19. The method of claim 14, further comprising the step of moistening the first layer prior to placing the neurosurgical sponge apparatus on the quantity of neuronal tissue.

20. A neurosurgical sponge apparatus comprising:
a moistened first layer formed from a fluid-retaining fabric;
a second layer affixed abutting to a first side of the first layer, wherein the second layer is formed from at least one water-soluble material; and
a low adhesion barrier formed by the second layer when the second layer is contacted by a quantity of neuronal tissue after a predetermined length of time.
